(19) United States
(12) Patent Application Publication
(10) Pub. No.: US 2026/0038652 A1
(43) Pub. Date: Feb. 5, 2026

(54) TELEHEALTH SUITE FOR PSYCHIATRY DIGITAL PHENOTYPING

(71) Applicant: The Johns Hopkins University, Baltimore, MD (US)

(72) Inventors: Erika K. Raskha, McLean, VA (US); Caroline Popper, Baltimore, MD (US); Crystal L. Butler, Ellicott City, MD (US); Mattson W. Ogg, Hyattsville, MD (US); Diego A. Luna, New York, NY (US); Rodrigo-Rene R. Munoz-Abujder, Laurel, MD (US); Han G. Yi, Ellicott City, MD (US); Hannah P. Cowley, Baltimore, MD (US); Peter Zandi, West Chester, PA (US)

(21) Appl. No.: 19/216,955

(22) Filed: May 23, 2025

Related U.S. Application Data
(60) Provisional application No. 63/678,308, filed on Aug. 1, 2024.

(51) Int. Cl. G16H 10/60; G16B 20/00; G16H 20/70; G16H 40/67; G16H 50/30

(52) U.S. Cl. CPC G16H 10/60; G16B 20/00; G16H 20/70; G16H 40/67; G16H 50/30

(57) ABSTRACT

Disclosed herein are system, method, and computer program product embodiments for improving for improving telemedicine (e.g., remote) interactions by capturing multiple types of data (e.g., audio, visual, textual), using a series of machine learning models to generate predictions from the data, and providing the predictions to a provider during the telemedicine interaction. One or more machine learning models may be utilized to generate intermediate representations of features extracted from audio, visual, and textual data. The data may be of a target individual involved in a remote interaction such as a telemedicine interaction, a job coaching session, or other scenario. The intermediate representations may be input to a machine learning model configured to generate a digital phenotype of the target individual. The digital phenotype may indicate a predicted diagnosis of the target individual, may indicate sub-clinical biomarkers of the target individual, as well as a projected trajectory of the predicted diagnosis.

### CROSS-REFERENCE TO RELATED APPLICATIONS

The present application claims priority to and filing benefit of U.S. Provisional Patent Application No. 63/678,308, filed on Aug. 1, 2024, which is incorporated herein by reference in its entirety.

### BACKGROUND

### Field

This field is generally related to utilizing a multimodal machine learning model to provide real-time patient information.

### Related Art

The rise of telemedicine platforms has increased the number of patients that are able to engage with physicians in online sessions to receive medical treatment or counseling. However, the physician or provider's ability to diagnose and identify the best treatment for the patient is dependent upon the provider performing an accurate assessment of the individual. In a telemedicine interaction, various factors may degrade the physician's ability to properly assess the patient. For instance, lack of physical contact, missing vital signs such as heart rate, and heavier reliance on verbal cues may lead to difficulties in diagnosing and treating a patient. Current psychiatric disorder study diagnostic evaluations typically rely upon finite interactions in artificial clinical settings. The lack of quantitative measures complicates detection of clinically relevant changes per patient.

### BRIEF SUMMARY

Disclosed herein are system, apparatus, device, method and/or computer program product embodiments, and/or combinations and sub-combinations thereof, for detecting and building a personalized digital phenotype of an individual via standoff sensing. In some embodiments, the personalized digital phenotype may be further based on integrated contact sensing data. The digital phenotype may be generated during a remote interaction (e.g., a telemedicine interaction) by capturing multiple types of data (e.g., audio, visual, textual, and/or wearable sensor data), using a series of machine learning models to generate predictions from the data, and providing the predictions to a provider during the remote interaction. The digital phenotype provides quantitative data on the individual's biomarkers and symptom state trajectories to: (1) inform a practitioner; and (2) provide an understanding of the individual's state over time.

Each machine learning model may be trained and configured to receive as input a specific data type (e.g., visual data) and generate a prediction regarding a target individual (e.g., the patient) from the data. Predictions from one or more machine learning models may be combined and input to a final machine learning model in the series. The final multimodal model may be configured to predict a digital phenotype of the target individual based at least on the predictions from the series of machine learning models. The digital phenotype may include a current diagnosis, a trajectory estimation, and/or one or more sub-type estimations (e.g., sub-clinical biomarkers). For example, the digital phenotype may predict a rating of the target individual on the Depressed, Anxious, Stressed, or Neutral (DASS) scale, an emotion estimate (e.g., happy, angry, sad, neutral, delighted, excited, tense, angry, frustrated, depressed, bored, tired, calm, relaxed, or content), or raw valence and arousal plots, or any combination thereof. The digital phenotype may be output by the system. For example, the digital phenotype may be transmitted to a computing device, displayed as a visual notification, or stored in memory for future access, such as within the individual's electronic health record.

The application space of the generated digital phenotype is not limited to telemedicine psychiatric interactions, as there are multiple application areas in medicine, job coaching, and other social-behavioral assessments which may find important use of its data. In some embodiments, the personalized digital phenotype may inform telehealth or in-person assessments of individuals with neurological conditions or developmental disorders, which may include but are not limited to: autism and neurological diseases such as amyotrophic lateral sclerosis (ALS), stroke, multiple sclerosis and seizure disorders such as epilepsy.

### BRIEF DESCRIPTION OF THE DRAWINGS

The accompanying drawings are incorporated herein and form a part of the specification.

FIG. 1 depicts a block diagram of an environment for determining a digital phenotype, according to some embodiments.

FIG. 2 depicts a block diagram for using multiple machine learning models to determine a digital phenotype, according to some embodiments.

FIG. 3 depicts a flowchart illustrating a method for generating a digital phenotype, according to some embodiments.

FIG. 4 depicts an example computer system useful for implementing various embodiments.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, device, method and/or computer program product embodiments, and/or combinations and sub-combinations thereof, for improving telemedicine interactions by capturing multiple types of data (e.g., audio, visual, textual), using a series of machine learning models to generate predictions from the data, and providing the predictions to a provider during the telemedicine interaction.

Current telemedicine systems allow for patients and medical providers (e.g., physicians, social workers, counselors, psychologists) to interact while located remotely from one another. For example, a patient may see their psychiatrist via a videoconferencing platform, as opposed to going to the psychiatrist's physical office. Similarly, a patient may interact with their internal medicine doctor via a telehealth session. While offering many benefits in terms of convenience, telemedicine interactions provide limited assessment via verbal and audio cues from the patient, lacking vital signs or sub-clinical biomarker information, as well as analysis of personal digital phenotype changes over time. These interactions rely on providers to interpret limited data (e.g., two-dimensional video, audio) of a patient without quantitative patient data.

To address such issues, systems and methods are disclosed that utilize standoff sensing made possible by machine learning and multi-modal data to augment a telemedicine communication session with data such as detected visual, audio, and textual features. Features may also be extracted from sensor or biometric data originating from a wearable sensor or a contact sensor. Features may include, but are not limited to, one or more of biometric (e.g., physiological) features (e.g., estimated heart rate); facial action unit intensity; valence and arousal pairs; emotion classification; image, prompt, and correlation feature vectors; head pose; body pose; or eye tracking estimation. Features may be determined or predicted based on visual, audio, textual, and sensor data of the patient. The machine learning model may generate an intermediate representation based on the extracted feature(s).

The intermediate representation including the extracted features may be input to a machine learning model to generate a digital phenotype. The digital phenotype may include, but is not limited to, one or more of: (1) a Depression Anxiety Stress Scales (DASS) estimate; (2) a Patient Health Questionnaire (PHQ-9) estimate; (3) a Generalized Anxiety Disorder (GAD-7) estimate; (4) an emotion estimate; (5) behavioral prediction; (6) distress warning signs; (7) sub-clinical biomarker estimates with anomalies indicated; or (8) any other clinical questionnaire estimate. The digital phenotype may further include a current diagnosis (e.g., a mood disorder), a trajectory estimation (represented as at least one of: a trendline of quantitative biomarker estimates or an associated interpretation statement (e.g., patient likely to experience upcoming depressive episode)), and a sub-type estimation. The digital phenotype may further include any of the extracted features above such as biometric features or the emotion classification. A DASS estimate may be a score based on a questionnaire configured to score depression, anxiety, and stress. A PHQ-9 estimate may be a score based on a questionnaire configured to estimate depression severity. A GAD-7 estimate may be a score based on a questionnaire configured to estimate anxiety severity. In some embodiments, the patient may not have filled out a DASS questionnaire, PHQ-9 questionnaire, GAD-7 questionnaire, or any combination thereof, prior to generation of the digital phenotype, and such information is instead provided by a model trained to identify such estimates.

The digital phenotype may be provided to the physician, patient, or both. Similarly, the digital phenotype may be stored in an electronic health record of the patient. This is beneficial to enable long-term tracking of the digital phenotype. For example, the system may be configured to automatically compare previously determined digital phenotypes to a current digital phenotype. In some embodiments, based on a difference between the previous and current digital phenotypes, the physician or patient may be notified. In some embodiments, the extracted features and/or the digital phenotype may be communicated to entities in addition to the provider. For example, the digital phenotype may be communicated to a hospital as part of the patient's medical records. Similarly, the digital phenotype may be provided to emergency medical services in a medical emergency.

The machine learning model may be a multimodal model, configured to receive as input different types of data (e.g., images and text). In some embodiments, the machine learning model may be retrained (e.g., updated). For example, if additional or new ground truth patient data becomes accessible—such as filled out clinical questionnaires or other electronic health record information newly updated—the model may be retrained live to adjust per person.

Conventional psychological or psychiatric diagnoses are based on subjective factors observed by the physician or counselor. However, it is often difficult for the provider to appreciably describe these factors in the patient's medical records because of their subjective nature. This problem may become acute when, for example, a patient switches practices and their medical records including the previous physician's notes are transferred to the new practice. The physician's notes within the medical records may be deficient. As a result, the new physician may be unable to fully appreciate the diagnosis determined by the previous physician. However, by generating and including extracted features and the digital phenotype within the medical record the new physician may have additional measures by which to judge the patient prior to and during treatment. In some embodiments, the digital phenotype may be based on information in the medical record such as previously collected vital signs, clinical observations, patient questionnaires, or any combination thereof. In some embodiments, the digital phenotype may be determined without reference to a medical record. In some embodiments, the digital phenotype may be determined based solely on the medical record.

While the disclosure describes embodiments in the context of telemedicine interactions, the embodiments are not limited to these embodiments. The systems and methods described may be used during other interactions where the participants are remote from each other, such as for job coaching an individual preparing for a job interview. Similarly, the systems and methods described may be used to monitor estimated fatigue of a target individual. Based on the estimated fatigue, the target individual, a third-party, or both may be alerted.

Various embodiments of these features will now be discussed with respect to the corresponding figures.

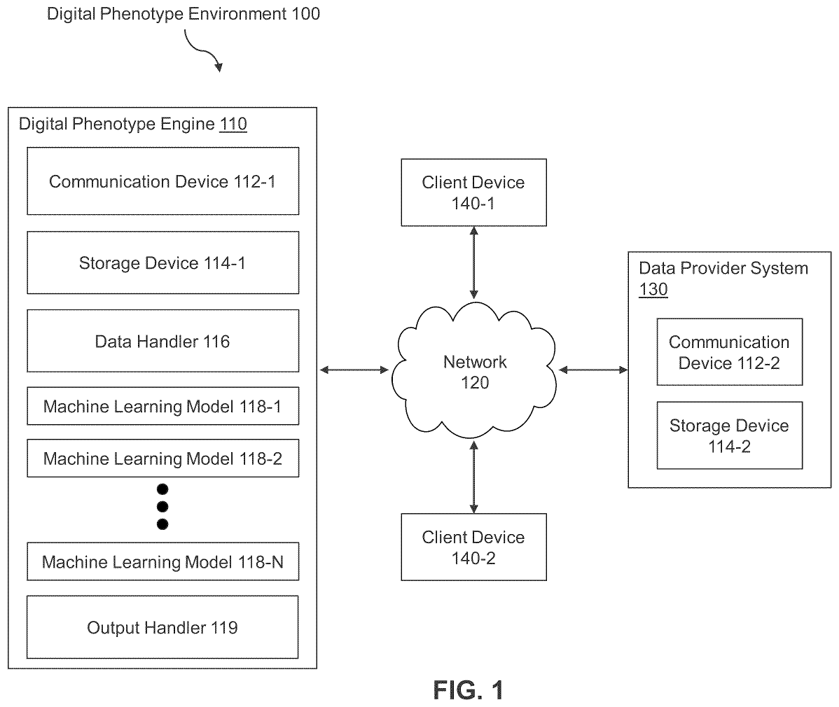

FIG. 1 depicts a block diagram of an environment 100 for determining a digital phenotype, according to some embodiments. Environment 100 includes digital phenotype engine 110, network 120, data provider system 130, and client device 140.

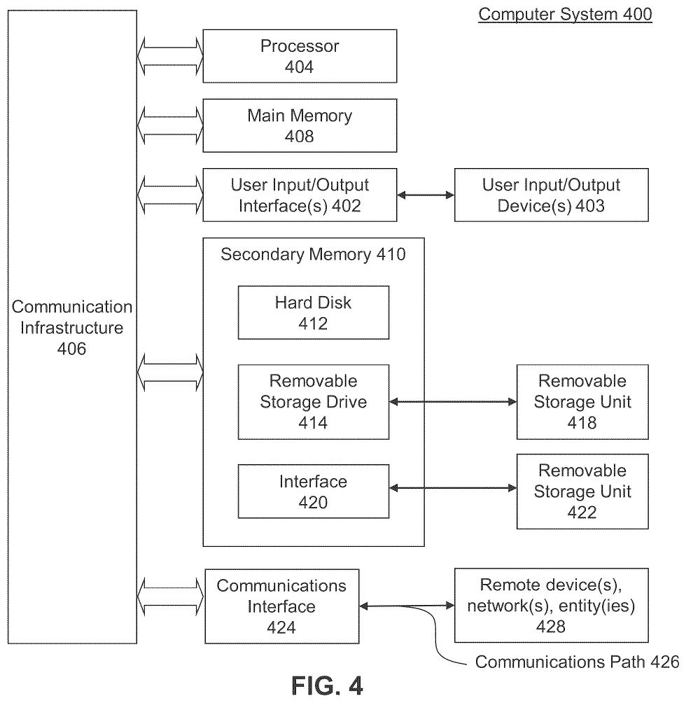

Digital phenotype engine 110 may be used to analyze data of a target individual (e.g., a patient) and generate a digital phenotype. Digital phenotype engine 110 may be implemented using a computing device such as a desktop workstation, laptop or notebook computer, netbook, tablet, smart phone, and/or other computing device. In some embodiments, digital phenotype engine 110 may be implemented as an application in an enterprise computing system, a cloud-computing system, a third-party electronic health record computing system, and/or a third-party electronic health record storage system. In some embodiments, digital phenotype engine 110 may be a computer system such as computer system 400 described with reference to FIG. 4. In some embodiments, digital phenotype engine 110 may be a software application. For example, digital phenotype engine 110 may be a plug-in integrated with a videoconferencing application. For example, a physician may use a video conferencing application and digital phenotype engine 110 in tandem to provide additional information to overcome the technological challenges presented by the patient being in a location that is remote from the physician. In some embodiments, digital phenotype engine 110 may be an application on a computing system connected to a video camera filming an in-person treatment session. As a result, digital phenotype engine 110 may analyze the audio and visual data captured by the video camera in real-time as it is recorded.

Digital phenotype engine 110 includes communication device 112-1, storage device 114-1, data handler 116, and output handler 119. Communication device 112-1 may include any suitable network interface capable of transmitting and receiving data, such as, for example a modem, an Ethernet card, a Wi-Fi antenna, a communications port, or the like. Communication device 112-1 may be able to transmit data using any wireless transmission standard such as, for example, Wi-Fi, Bluetooth, cellular, or any other suitable wireless transmission. Digital phenotype engine 110 may use communication device 112-1 to communicate with entities connected via network 120. In some embodiments, digital phenotype engine is directly connected to either or both of client device 140-1 or client device 140-2 rather than through network 120.

Network 120 may be any type of computer or telecommunications network capable of communicating data, for example, a local area network, a wide-area network (e.g., the Internet), or any combination thereof. The network may include wired and/or wireless segments.

Digital phenotype engine 110 may use communication device 112-1 to receive data from data provider system 130. Similarly, digital phenotype engine 110 may use communication device 112-1 communicate with client device 140 via network 120. As will be discussed below, a physician and a target individual (e.g., a patient) may engage in a telemedicine interaction using client device 140-1 and client device 140-2. Audio and visual data transmitted as part of the telemedicine communication may be received at communication device 112-1 of digital phenotype engine 110 for processing.

Storage device 114-1 may be any memory storage device. Digital phenotype engine 110 may use storage device 114-1 to store data of a telemedicine communication session, settings data, and/or data of the target individual. For example, one or more health records of the target individual involved in a telemedicine interaction may be stored at storage device 114-1. Storage device 114-1 may also be used to store one or more of machine learning models 118-1-118-N.

Data received at communication device 112-1 of digital phenotype engine 110 may be input to data handler 116. In some embodiments, data received at communication device 112-1 may be part of a data stream. Noted above, communication device 112-1 may receive audio and visual data generated during a telemedicine communication session. Here, communication device 112-1 may provide to data handler 116 the audio and visual data. As will be discussed below, digital phenotype engine 110 may further have access to textual data of the target individual such as their health records. Here, the textual data may also be input to data handler 116.

Data handler 116 may be configured to provide data to one or more machine learning models 118. Digital phenotype engine 110 may include any number of machine learning models 118 (e.g., machine learning model 118-1 to machine learning model 118-N). Although the disclosure below refers to “machine learning model 118” for clarity and brevity, a skilled artisan would recognize that aspects attributed to machine learning model 118 may apply to any or all of machine learning models 118-1-118-N. Machine learning model 118 may be trained using any type of data and have any architecture. For example, machine learning model 118 may be trained using a zero-shot contrastive feature mapping method. Machine learning model 118 may be constructed as, for example but not limited to, one or more of a linear regression model, a logistic regression model, a decision tree model, a support vector machine, a naïve Bayes model, a K-means model, a random forest model, a dimensionality reduction algorithm, a gradient boosting algorithm, a neural network, a deep neural network, a convolutional attention network, a transformer model, or a gated recurrent unit.

In some embodiments, machine learning model 118 may be one or more of, for example and without limitation: a deep neural network regressor model configured to estimate facial action units and arousal and valence pairs; a multi-task temporal shift attention network to estimate heart rate and heart rate variability; a deep learning model configured to estimate emotion classification from audio data; a deep learning model including one or more transformers to generate text-based prompts from textual data; or a deep learning model configured to estimate movement of the target individual including pose estimation (e.g., head pose, body pose).

In some embodiments, each machine learning model 118 may be trained to receive as input a type of data, extract a feature from the data, and generate an intermediate representation of the extracted feature. Data types may include, but are not limited to, visual data (e.g., image, video), audio data, textual data, and sensor data. Visual and audio data may originate from a telemedicine session between a physician and the patient. In some embodiments, the visual and audio data may be received in real-time during the telemedicine session. In some embodiments, digital phenotype engine 110 may receive a recording of a telemedicine session including visual and audio data. Textual data may be data from a health record of the patient including information such as the patient's medical history and physician notes. Textual data may include audio transcribed from a previous telemedicine communication session. In some embodiments, the textual data may be a live transcription of audio data generated during a current telemedicine interaction. Textual data may also include an emotional rating reported by the target individual. Sensor data may be data generated by a contact sensing or wearable sensor of the target individual. For example, the target individual may be wearing a sensor collecting biometric data such as heart rate, respiratory rate, temperature, heart rate variation, blood oxygen levels, and blood pressure. In some embodiments, sensor data may further include location information.

In some embodiments, data handler 116 may input a specific type of data to machine learning model 118. For example, data handler 116 may receive an image formatted as a JPEG or PNG and provide the image to machine learning model 118, where machine learning model 118 is configured to receive image data. In some embodiments, data handler 116 may perform a data processing process whereby multiple types of data are extracted from a single input. For example, digital phenotype engine 110 may receive, as input, video and audio data stored within an .mp4 file. Here, data handler 116 may be configured to extract the audio and video data from the input. This is beneficial so that data handler 116 may route the input data types to machine learning model 118 configured to receive the input data type. For example, data handler 116 may route the audio data to machine learning model 118-1, where machine learning model 118-8 is configured to process audio data, and the video data to machine learning model 118-2, where machine learning model is configured to process video data. Data handler 116 may be further configured to track and add time information to received data. Time information may relate to a date and time that the received data was generated. For example, an image may have a timestamp of when the image was taken. Similarly, an input stream including both audio and visual data may have one or more timestamps indicating the time that the audio and visual data was captured. Biometric data (e.g., heart rate) may also have timestamps corresponding to when the biometric data was captured.

In some embodiments, data received by data handler 116 may already include time data. In some embodiments, data handler 116 may add time information to received data. For example, data handler 116 may extract audio data and visual data from a received .mp4 file. Data handler 116 may add the timestamp data from the .mp4 file to both the extracted audio data and extracted visual data. As a result, data handler 116 may provide data to machine learning model 118 in a time synchronized manner. For example, audio, visual, and biometric data captured at the same time, or within a predefined time window may be grouped and input to a machine learning model 118 for analysis.

As noted above, machine learning model 118 may be configured to receive an input and extract one or more features from the input. Machine learning model 118 may be configured to receive a specific type of input such as audio, visual, biometric, or text data. In some embodiments, machine learning model 118 may be multi-modal and be configured to receive multiple types of data such as audio and visual data. In some embodiments, digital phenotype engine 110 may include a single instance of machine learning model 118 including multiple layers. Each layer may be configured to a specific type of data (e.g., audio data, visual data, textual data, or biometric data). In some embodiments, machine learning model 118 may be a statistical model.

Machine learning model 118 may be configured to extract features based on received data including, but not limited to, one or more of: (1) a face of the target individual from visual data; (2) a body of the target individual from visual data; (3) an emotional affect of the target individual from visual or audio data; (4) a voice prosody of the target individual from audio data; (5) a heart rate of the target individual from visual data; (6) a raw blood volume pulse signal of the target individual from visual data; (7) a heart rate variability of the target individual from visual data; (8) an output time series forecast; or (9) a data imputation. An output time series forecast may be a predicted future value of patient data. In some embodiments, the output time series forecast may be a single value. In some embodiments, the output time series forecast may include multiple values. For example, the output time series forecast may be a series of predicted heart rate values over the next 30 seconds. A data imputation may be a predicted value that is used to replace missing data. In some embodiments, the data imputation may be used as input to machine learning model 118.

In some embodiments, a single machine learning model 118 may extract the features based on the received data. In some embodiments, multiple machine learning models 118 may extract the features based on the received data.

Machine learning model 118 may be configured to receive as input one or more of the features listed above to predict the digital phenotype. The digital phenotype may be a predicted diagnosis of the individual. The digital phenotype may include an estimated trajectory of the predicted diagnosis. For example, the digital phenotype may indicate the target individual has major depression disorder and is likely to experience a major depressive episode. The digital phenotype may include any of the features listed above (e.g., emotional affect, heart rate, and heart rate variability). The digital phenotype may further include a DASS estimated score, PHQ-9, estimated score, and/or GAD-7 estimated score. Furthermore, the digital phenotype may include a predicted emotion such as happy, angry, sad, neutral, delighted, excited, tense, angry, frustrated, depressed, bored, tired, calm, relaxed, or content. In some embodiments, machine learning model 118 may include a confidence score (e.g., 90%) within the digital phenotype. The confidence score may correspond to a confidence level of machine learning model 118 that the digital phenotype is correct. In some embodiments, machine learning model 118 may include a confidence score to each item of the digital phenotype. For example, the digital phenotype may include a predicted diagnosis, an estimated trajectory of the predicted diagnosis, an emotional affect, and heart rate variability. Machine learning model 118 may include a confidence score for some or all of these items.

In some embodiments, machine learning model 118 may be configured to predict the digital phenotype based on a limited or single input. For example, client device 140-1 may be a wearable sensor of the target individual configured to generate and send biometric data to digital phenotype engine 110. Digital phenotype engine 110 may utilize machine learning model 118 to generate a digital phenotype based only on the biometric data. This is beneficial to continuously provide digital phenotype information outside of a telemedicine visit. In some embodiments, digital phenotype engine 110 may reference previously generated digital phenotypes to predict a future digital phenotype. For example, in a scenario where digital phenotype engine 110 only receives biometric data, it may retrieve a previously generated digital phenotype of the target individual, and input both the biometric data and previously generated digital phenotype to machine learning model 118 to generate a current digital phenotype.

Output handler 119 of digital phenotype engine 110 may utilize the digital phenotype output by machine learning model 118 in various ways. For example, output handler 119 may add the digital phenotype to the patient's health record stored in memory at storage device 114-1. Similarly, output handler 119 may transmit the digital phenotype for storage at an entity responsible for maintaining the patient's health record, such as data provider system 130. Output handler 119 may be further configured to provide the digital phenotype as a visual notification within a graphical user interface (GUI) located either locally or remotely. As discussed above, a physician and patient may each utilize client device 140-1 and client device 140-2 during a telemedicine communication session. Digital phenotype engine 110 may receive data of the telemedicine communication session, determine the patient's digital phenotype, and use output handler 119 to transmit the digital phenotype to either or both client device 140-1 and client device 140-2. The digital phenotype may be displayed within a GUI at client device 140. For example, the digital phenotype may be displayed as a visual notification (e.g., a popup) within the GUI. Output handler 119 may be further configured to transmit the digital phenotype to a device via network 120. For example, output handler 119 of digital phenotype engine 110 may transmit the digital phenotype to a client device 140 associated with a hospital or emergency services. Similarly, output handler 119 may transmit the digital phenotype to a remote display platform.

In some embodiments, digital phenotype engine 110 may include a GUI configured to display the digital phenotype and one or more extracted features. For example, output handler 119 may display within a GUI the estimated biometric data of the target individual over time. Similarly, output handler 119 may graph the occurrences of one or more emotions of the target individual over time. For example, output handler 119 may plot the number of times a digital phenotype has indicated that the target individual experienced a depressive episode during the previous six months. Similarly, output handler 119 may plot a projected trajectory of the patient's diagnosis, such as whether they are likely to experience a depressive episode or manic episode in the coming weeks.

As noted above, the digital phenotype may include one or more confidence scores. Digital phenotype engine 110 may be further configured to display the confidence scores of the digital phenotype. For example, the digital phenotype may include a predicted heart rate of 60 beats per minute (BPM) with a corresponding confidence score of 85%. Digital phenotype engine 110 may display at the GUI: 60 BPM; 60%. In some embodiments, digital phenotype engine 110 may only display confidence scores less than or equal to a predefined threshold (e.g., 70%). This is beneficial because it allows the recipient of the digital phenotype (e.g., the physician) to determine how much to rely on the information. For example, if the digital phenotype includes a predicted heart rate with 30% confidence, the physician may take this into account when determining a diagnosis and/or treatment for the patient. Selectively displaying the confidence score is also beneficial to prevent the GUI from becoming cluttered and distracting the viewer (e.g., the physician). In some embodiments, digital phenotype engine 110 may not display information of the digital phenotype if it is below a predefined threshold (e.g., 30%). As noted above, digital phenotype engine 110 may add the digital phenotype to the patient's health record. In some embodiments, digital phenotype engine 110 may only add information of the digital phenotype to the health record if it has a corresponding confidence score greater than or equal to a predefined threshold. This is beneficial to prevent inaccurate data from being added to the health record and used to provide an incorrect diagnosis or treatment by a provider.

In some embodiments, digital phenotype engine 110 may be configured to display information at the GUI regarding input data. As noted above, digital phenotype engine 110 may receive audio, visual, textual, and sensor data. In some embodiments, digital phenotype engine 110 may be unable to process the data. For example, machine learning model 118 may be unable to detect a human voice within the audio data. Similarly, machine learning model 118 may be unable to detect a face within the visual data. Digital phenotype engine 110 may be configured to display warnings at the GUI indicating errors regarding data processing. For example, digital phenotype engine 110 may display a warning that the face of the patient is undetectable in the video feed. In some embodiments, digital phenotype engine 110 may display feedback to improve the input data feed. For example, machine learning model 118 may be trained to analyze visual data and predict one or more actions to improve the quality. For example, machine learning model 118 may detect that low ambient light in an image that is degrading the image quality. Machine learning model 118 may provide an indication to digital phenotype engine 110 that the ambient light should be increased. Digital phenotype engine 110 may display a popup on the GUI stating that the ambient light at the source of the image should be increased. This will not only improve the interaction between the provider and the patient, but it will also improve the accuracy of the digital phenotype because the quality of data input to digital phenotype engine 110 will be improved.

Providing the digital phenotype to the physician at client device 140 allows for real-time diagnosis. Additionally, the digital phenotype accounts for factors that a physician may previously have been unable to utilize in their assessment during a telemedicine interaction because they were not physically located near the patient. As noted above, machine learning model 118 may predict biometric data of the target individual such as heart rate and heart rate variability. The physician may incorporate this information into their assessment and diagnosis of the individual. For example, the physician may suspect, based on how the patient looks and sounds, that they are experiencing a panic attack. However, the physician may be uncertain based on the quality of video or audio data of the telemedicine interaction. By receiving predicted biometric data such as a heart rate and/or a heart rate variability, the physician may use this predicted biometric data to confirm their diagnosis. For example, the biometric data may indicate that the patient has an elevated heart rate. The physician may determine that an elevated heart rate is associated with a panic attack, and subsequently confirm the patient is likely experiencing a panic attack. A similar scenario may occur where a patient shows no clear signs of distress, but based on facial affect changes over time and other data estimated by machine learning model 118, machine learning model 118 model may accurately predict that the patient is likely to experience an imminent depressive episode. This may be detected based on quantitative trends or other faint changes in the patient. As a result, the prediction may be generated and acted upon without solely relying upon the observations by the practitioner.

Data provider system 130 may be an entity capable of generating, storing, and transmitting data regarding the target individual. Data provider system 130 may be implemented using a computing device such as a desktop workstation, laptop or notebook computer, netbook, tablet, smart phone, and/or other computing device. In some embodiments, data provider system 130 may be implemented as an application in an enterprise computing system and/or a cloud-computing system. In some embodiments, data provider system 130 may be a computer system such as computer system 400 described with reference to FIG. 4. For example, data provider system 130 may be affiliated with a hospital or other medical provider and used to store medical records or other data of the target individual. Data provider system 130 may respond to data requests from digital phenotype engine 110, client device 140, or both. For example, a physician may use client device 140-2 to access and view patient records stored at data provider system 130. Data provider 130 may include a communication device 112-2 and a storage device 114-2, which share similar features as communication device 112-1 and storage device 114-1, respectively.

Client device 140 may be any device configured to interact with digital phenotype engine 110 and data provider system 130 via network 120. Client device 140 may be implemented using a computing device such as a desktop workstation, laptop or notebook computer, netbook, tablet, smart phone, and/or other computing device. In some embodiments, client device 140 may be implemented as an application in an enterprise computing system and/or a cloud-computing system. In some embodiments, client device 140 may be a computer system such as computer system 400 described with reference to FIG. 4. Although environment 100 depicts two instances of client device 140, client device 140-1 and client device 140-2, environment 100 may include one or any number of client devices 140.

For example, client device 140-1 may be used by a patient as part of a telemedicine interaction. Similarly, the physician may use client device 140-2 to interface with the patient during the telemedicine interaction. Client device 140 may include a software application such as a videoconferencing application to support the telemedicine interaction. Here, data transmitted between client device 140-1 and client device 140-2 may be routed on network 120 through digital phenotype engine 110 for analysis. As noted above, digital phenotype engine 110 may be implemented as a software application (e.g., a plugin). Here, client device 140 may include an instance of digital phenotype engine 110 to analyze data that is received at and sent by client device 140.

In some embodiments, client device 140 may be a wearable sensor (e.g., heart rate monitor, smart watch) that captures biometric data of the target individual. The biometric data may be provided to digital phenotype engine 110 for use in generating the digital phenotype. In some embodiments, client device 140 may belong to a third party not involved in a telemedicine interaction. For example, digital phenotype engine 110 may generate a digital phenotype including indication of early distress warning signs. In some embodiments, based on factors such as the target individual's occupation or the details of the early warning signs, the digital phenotype may be reported to the target individual's employer or emergency services (e.g., when permissions are in place or when there is a medical emergency). Client device 140 may be associated with the target individual's employer or emergency services and receive the digital phenotype generated by digital phenotype engine 110. In some embodiments, digital phenotype engine 110 may require permission from client device 140 of the target individual prior to transmitting the digital phenotype to a third party (e.g., an employer).

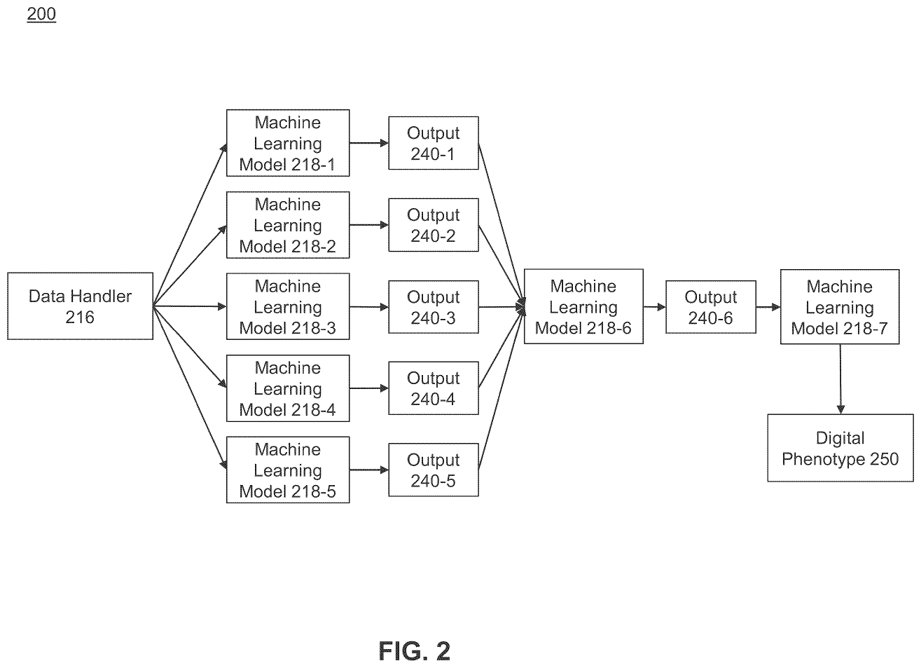

FIG. 2 depicts a block diagram of architecture 200 for using multiple machine learning models to determine a digital phenotype, according to some embodiments. Architecture 200 includes data handler 216, machine learning model 218, output 240, and digital phenotype 250. Data handler 216 may be the same as data handler 116 described with respect to FIG. 1. Machine learning model 218 may be the same as machine learning model 118 described with respect to FIG. 1.

As discussed above, data handler 216 may receive data as part of a telemedicine (e.g., telehealth) session including visual and audio data. Data handler 216 may also receive sensor data from a contact sensing or wearable device of the patient. Data handler 216 may further receive textual data such as a health record of the patient. As depicted in architecture 200, data handler 216 may provide data to machine learning model 218. Machine learning model 218 may include several individual machine learning models. According to some embodiments, FIG. 2. shows machine learning models 218-1 through 218-5. However, architecture 200 may include more or fewer machine learning models in other embodiments. Each machine learning model referenced herein may include one or more machine learning models that individually or in combination (e.g., in sequence or in parallel) generate a noted output.

Data handler 216 may send the same data to each machine learning model 218, different data to each machine learning model 218, or any combination thereof. For example, machine learning model 218-1 may be configured to process visual data and machine learning model 218-2 may be configured to process audio data. As a result, data handler 216 may provide visual data to machine learning model 218-1 and audio data to machine learning model 218-2.

Each machine learning model 218 may be configured to generate an output 240. In FIG. 2, each machine learning model 218-1-218-5 generates a respective output 240-1-240-5. Output 240 may be a feature extracted from the data input to corresponding machine learning model 218. Output 240 may depend on the data input to corresponding machine learning model 218 and the architecture of corresponding machine learning model 218.

Machine learning model 218-1 may be configured as at least one of: a neural network configured to perform a neural network method, a deep neural network (DNN), or a transformer model. Machine learning model 218-1 may be configured to receive and process visual data, and output 240-1 may be extracted data representing a specific aspect of the target individual. For example, output 240-1 may be extracted data representing a face (e.g., an image and/or position of the face) of the target individual or extracted data representing a body (e.g., an image and/or position of the body) of the target individual. An example deep learning machine learning model that can be used in a customized model to generate such data is the OpenFace toolkit as described in B. Amos et al., “Openface: A general-purpose face recognition library with mobile applications,” CMU-CS-16-118, CMU School of Computer Science, Tech. Rep., 2016.

Machine learning model 218-2 may be configured as at least one of: a neural network configured to perform a neural network method, a deep neural network (DNN), or a transformer model. For example, machine learning model 218-2 may use a DNN regressor model. Machine learning model 218-2 may be configured to receive and process visual data. Output 240-2 may include an intermediate representation of an emotional affect of the target individual using the visual data or an image or other data representation of the body of the target individual using the visual data. Output 240-2 may further include an output estimation of a facial action unit intensity or a valence and arousal pair estimation.

Machine learning model 218-3 may be configured to receive and process audio data. Machine learning model 218-3 may be configured as at least one of: a neural network configured to perform a neural network method, a deep neural network, or a transformer model. Output 240-3 may include an intermediate representation of a voice of the target individual. Output 240-3 may further include an output estimation of an emotion classification or a voice prosody feature. An example deep learning machine learning model that may be used to generate such data is the Self-Supervised Speech Pre-training and Representation Learning (“s3prl”) toolkit as described by A. T. Liu et al., “TERA: Self-Supervised Learning of Transformer Encoder Representation for Speech,” IEEE/ACM Transactions on Audio, Speech, and Language Processing, vol. 29, pp. 2351-2366, 2021 (see also A. Liu et al., “Mockingjay: Unsupervised Speech Representation Learning with Deep Bidirectional Transformer Encoders,” IEEE International Conference on Acoustics, Speech and Signal Processing, pp. 6419-6423, 2019.)

Machine learning model 218-4 may be configured to receive and process visual data. Machine learning model 218-4 may be configured as a neural network configured to perform a neural network method, a deep neural network, a transformer model, or any combination thereof. For example, machine learning model 218-4 may include a multi-task temporal shift attention network. Output 240-4 may include an output estimation of biometric data such as a heart rate of the target individual, a raw blood volume pulse signal of the target individual, and/or a heart rate variability of the target individual. Machine learning model 218-4 may be configured generate output 240-4 including the estimation of biometric data based on an intermediate representation of the face of the target individual or an intermediate representation the body of the target individual. For example, machine learning model 218-4 may reference a feature depicted in an intermediate representation of the target individual's face to estimate heart rate. In some embodiments, output 240-4 may include the intermediate representation of the face of the target individual or the body of the target individual. An example deep learning machine learning model that can be used in a customized model to generate such data is the MTTS-CAN remote photoplethysmography (“rPPG”) toolkit as described in X. Liu et al., “Multi-Task Temporal Shift Attention Networks for On-Device Contactless Vitals Measurement,” 34^{th} Conference on Neural Information Processing Systems, 2020.

Machine learning model 218-5 may be configured to receive and process visual data or textual data, such as data from a health record (e.g., an electronic medical record). Machine learning model 218-5 may be configured as a neural network configured to perform a neural network method, a deep neural network, a transformer model, or any combination thereof. Machine learning model 218-5 may be further configured to execute a zero-shot contrastive pre-training method. Such a zero-shot contrastive pre-training method may be used, for example, for feature mapping. An example zero-shot contrastive pre-training tool may be customized for feature mapping based on the GLORIA framework as described in S. C. Huang et al., “GLORIA: A Multimodal Global-Local Representation Learning Framework for Label-Efficient Medical Image Recognition,” 2021 Proceedings of the IEEE/CVF International Conference on Computer Vision, pp. 3942-3951, 2021. Output 240-5 may include an intermediate representation of a health record of the patient. For example, the intermediate representation may include the patient's medical history written in the health record, or the patient's historical biometric data written in the health record. In some embodiments, the intermediate representation may be an image and prompt correlation of one or both of: (1) the health record of the target individual using the visual and text data; or (2) the biometric data using the visual and text data.

Machine learning model 218-6 may be a neural network configured to perform a neural network method, a recurrent neural network-based model, a transformer model configured to employ a probabilistic forecasting method or a self-attention method, a large language model, or any combination thereof. An example transformer model that can be customized to employ a probabilistic forecasting method is the ProbSparse model as described in H. Zhou et al., “Informer: Beyond Efficient Transformer for Long Sequence Time-Series Forecasting,” 35^{th} AAAI Conference on Artificial Intelligence, vol. 35, no. 12, pp. 11106-11115, 2021. Machine learning model 218-6 may be configured to receive output 240. For example, machine learning model 218-6 may be configured to receive, as input, one or more of output 240-1, output 240-2, output 240-3, output 240-4, or output 240-5. Machine learning model 218-6 may be configured to generate output 240-6 based on the received input. Output 240-6 may be an output time series forecast or a data imputation. For example, output 240-6 may include each preceding output 240 (e.g., output 240-1 to 240-5) with a respective time series forecast. For example, output 240-4 may include an output estimation of biometric data such as a heart rate of the target individual. Machine learning model 218-6 may predict a time series forecast for the target individual's heart rate. As a result, output 240-6 may include output 240-4 (e.g., the predicted current heart rate) and a time series forecast heart rate (e.g., the predicted future heart rate). Machine learning model 218-6 may generate output 240-6 by applying a weight or a transform logic to the received input.

A final machine learning model 218, such as machine learning model 218-7, may be configured to receive, as input, output 240-6 and generate digital phenotype 250. Machine learning model 218-7 may be configured to perform a neural network method, a statistical method, control logic methods, or any combination thereof. In some embodiments, machine learning model 218-7 may be further configured to receive as input and use output one or more of 240-1, output 240-2, output 240-3, output 240-4, or output 240-5 in generating digital phenotype 250.

As noted above, digital phenotype 250 may be include a predicted diagnosis of the individual. Digital phenotype 250 may include an estimated trajectory of the predicted diagnosis. For example, digital phenotype 250 may indicate the target individual has major depression disorder and is likely to experience a major depressive episode. Digital phenotype 250 may be provided in any communicative format, such as text and/or images. Digital phenotype 250 may include one or more of a natural language text description, a DASS estimated score, a PHQ-9, estimated score, or a GAD-7 estimated score. Furthermore, digital phenotype 250 may include a predicted emotion such as happy, angry, sad, neutral, delighted, excited, tense, angry, frustrated, depressed, bored, tired, calm, relaxed, or content. Digital phenotype 250 may further include an extracted feature such as a facial action unit intensity or estimated biometric data.

Digital phenotype 250 may be provided to the physician and/or the patient involved in the telemedicine interaction. Digital phenotype 250 may additionally be added to the patient's health record. This is beneficial because the physician can use digital phenotype 250 as an additional data point indicative of the patient's health, similar to a patient's BMI or cholesterol levels. In some embodiments, output handler 119 of digital phenotype engine 110 may transmit digital phenotype 250 to a hospital or emergency services if digital phenotype 250 indicates that the patient is exhibiting early warning signs of certain distress such as suicidal ideation.

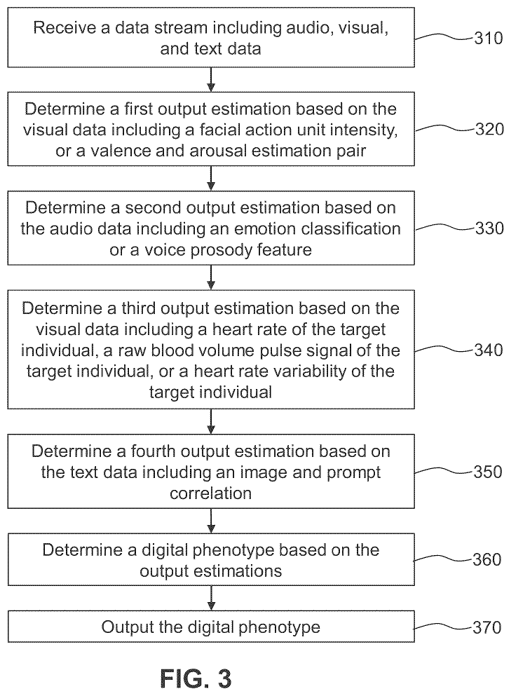

FIG. 3 depicts a flowchart illustrating a method 300 for generating a digital phenotype, according to some embodiments. Method 300 shall be described with reference to FIG. 1, however, method 300 is not limited to that example embodiment.

In an embodiment, digital phenotype engine 110 may utilize method 300 to generate a digital phenotype. The digital phenotype may be based on one or more features extracted from audio, visual, textual, and biometric data. In some embodiments, digital phenotype engine 110 may generate the digital phenotype in real-time during a telemedicine communication session between a physician and a target individual (e.g., a patient). The data utilized to generate the digital phenotype may be generated during the telemedicine communication session. For example, the audio and visual data may be data of the telemedicine communication session between the physician and target individual. The textual data maybe a transcription of the telemedicine communication session. In some embodiments, the textual data may be a health record of the target individual. The health record may be retrieved from a storage system such as storage device 114-2 of data provider system 130 via communication device 112-2. The biometric data may be generated by a wearable sensor of the target individual such as a smart watch including a heart rate monitor. In some embodiments, digital phenotype engine 110 may predict biometric data of the target individual based on visual data and/or audio data.

The foregoing description will describe an embodiment of the execution of method 300 with respect to digital phenotype engine 110, which may be located as an instance on any client device 140 or located remotely therefrom. While method 300 is described with reference to digital phenotype engine 110, method 300 may be executed on any computing device, such as, for example, the computer system described with reference to FIG. 4 and/or processing logic that may include hardware (e.g., circuitry, dedicated logic, programmable logic, microcode, etc.), software (e.g., instructions executing on a processing device), or a combination thereof. For example, method 300 may be executed on client device 140, such as client device 140-2 associated with a physician.

It is to be appreciated that not all steps may be needed to perform the disclosure provided herein. Further, some of the steps may be performed simultaneously, or in a different order than shown in FIG. 3.

At 310, digital phenotype engine 110 receives a data stream including audio, visual, and text data. The data stream may be of a target individual, such as a patient during a telemedicine interaction. The data stream may be generated in real time as the telemedicine interaction occurs. In some embodiments, the data stream may be a recording. The audio data may be audio of the target individual speaking. The visual data may include images and video of the target individual. Textual data may be data from a health record of the patient including information such as the patient's medical history and physician notes. Textual data may include audio transcribed from a previous telemedicine communication session. In some embodiments, the textual data may be a live transcription of audio data generated during a current telemedicine interaction. Textual data may also include an emotional rating reported by the target individual, the health history of the target individual, physician notes, previous biometric data of the target individual, and previously generated digital phenotypes of the target individual. In some embodiments, the data stream may include additional data, such as sensor data generated by a wearable sensor in contact with the target individual.

At 320, digital phenotype engine 110 determines a first output estimation based on the visual data. The first output estimation may include a facial action unit intensity, or a valence and arousal estimation pair. The first output estimation may further include data representing the face of the target individual, data representing the body of the target individual, or data representing a pose of the target individual. Digital phenotype engine 110 may use one or more machine learning models, such as machine learning models 218-2, to determine the first output estimation.

At 330, digital phenotype engine 110 determines a second output estimation based on the audio data. The second output estimation may include an emotion classification or a voice prosody feature. Digital phenotype engine 110 may use one or more machine learning models, such as machine learning models 218-3, to determine the second output estimation.

At 340, digital phenotype engine 110 determines a third output estimation based on the visual data. The third output estimation may include a heart rate of the target individual, a raw blood volume pulse signal of the target individual, or a heart rate variability of the target individual. Digital phenotype engine 110 may use one or more machine learning models, such as machine learning models 218-4, to determine the third output estimation.

At 350, digital phenotype engine 110 determines a fourth output estimation based on the text data. The fourth output estimation may include an image and prompt correlation. Digital phenotype engine 110 may use one or more machine learning models, such as machine learning models 218-5, to determine the fourth output estimation.

At 360, digital phenotype engine 110 determines a digital phenotype based on the output estimations. The digital phenotype may be digital phenotype 250, discussed above. Digital phenotype engine 110 may use one or more machine learning models, such as machine learning models 218-6 and 218-7, to determine digital phenotype 250, wherein the one or more machine learning models uses outputs 240 as inputs. Noted above, the digital phenotype may include (1) a DASS estimate; (2) a PHQ-9 estimate; (3) a GAD-7 estimate; (4) an emotion estimate; (5) a behavioral prediction; and (6) distress warning signs. The digital phenotype may further include any of the extracted visual, audio, or textual features. For example, the digital phenotype may further include the estimated biometric data based on the visual data.

At 370, digital phenotype engine 110 outputs the digital phenotype. Digital phenotype engine 110 may output the digital phenotype using output handler 119. For example, output handler 119 may transmit the digital phenotype for display at a computing device of the physician (e.g., client device 140-2). Similarly, output handler 119 may transmit the digital phenotype as a notification to a hospital or emergency services entity. Output handler 119 may further store the digital phenotype within the health record of the target individual. For example, digital phenotype engine 110 may receive the health record of the target individual from data provider system 130, determine a digital phenotype, add the digital phenotype to the health record, and transmit the updated health record to data provider system 130.

In some embodiments, digital phenotype engine 110 may determine the digital phenotype without certain data types of the data stream. For example, digital phenotype engine 110 may determine the digital phenotype based on visual and audio data (e.g., without textual data).

While digital phenotype engine 110 has been described with respect to a telehealth or telemedicine interaction, digital phenotype engine 110 may be utilized during other interactions, such as a coaching session for a neurodiverse population. For example, the generated digital phenotype may be displayed as textual or visual guidance for socio-behavioral learning or job coaching.

While digital phenotype engine 110 has been described with respect to a telehealth or telemedicine interaction, digital phenotype engine 110 may be utilized during a telehealth or in-person assessment of individuals with neurological or developmental disorders/conditions. For example, anomaly detection algorithms within the engine may serve to detect if facial patterns have significantly differed over a shorter than baseline-typical period of time. This may support autism or neurological diseases recognition and/or support, such as amyotrophic lateral sclerosis (ALS), stroke, multiple sclerosis and seizure disorders such as epilepsy.

Various embodiments may be implemented, for example, using one or more computer systems, such as computer system 400 shown in FIG. 4. One or more computer systems 400 may be used, for example, to implement any of the embodiments discussed herein, as well as combinations and sub-combinations thereof.

Computer system 400 may include one or more processors (also called central processing units, or CPUs), such as a processor 404. Processor 404 may be connected to a communication infrastructure or bus 406.

Computer system 400 may also include user input/output device(s) 403, such as monitors, keyboards, pointing devices, etc., which may communicate with communication infrastructure 406 through user input/output interface(s) 402.

One or more of processors 404 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 400 may also include a main or primary memory 408, such as random access memory (RAM). Main memory 408 may include one or more levels of cache. Main memory 408 may have stored therein control logic (e.g., computer software) and/or data.

Computer system 400 may also include one or more secondary storage devices or memory 410. Secondary memory 410 may include, for example, a hard disk drive 412 and/or a removable storage device or drive 414. Removable storage drive 414 may be a floppy disk drive, a magnetic tape drive, a compact disk drive, an optical storage device, tape backup device, and/or any other storage device/drive.

Removable storage drive 414 may interact with a removable storage unit 418. Removable storage unit 418 may include a computer usable or readable storage device having stored thereon computer software (control logic) and/or data. Removable storage unit 418 may be a floppy disk, magnetic tape, compact disk, DVD, optical storage disk, and/any other computer data storage device. Removable storage drive 414 may read from and/or write to removable storage unit 418.

Secondary memory 410 may include other means, devices, components, instrumentalities or other approaches for allowing computer programs and/or other instructions and/or data to be accessed by computer system 400. Such means, devices, components, instrumentalities or other approaches may include, for example, a removable storage unit 422 and an interface 420. Examples of the removable storage unit 422 and the interface 420 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 400 may further include a communication or network interface 424. Communication interface 424 may enable computer system 400 to communicate and interact with any combination of external devices, external networks, external entities, etc. (individually and collectively referenced by reference number 428). For example, communication interface 424 may allow computer system 400 to communicate with external or remote devices 428 over communications path 426, which may be wired and/or wireless (or a combination thereof), and which may include any combination of LANs, WANs, the Internet, etc. Control logic and/or data may be transmitted to and from computer system 400 via communication path 426.

Computer system 400 may also be any of a personal digital assistant (PDA), desktop workstation, laptop or notebook computer, netbook, tablet, smart phone, smart watch or other wearable, appliance, part of the Internet-of-Things, and/or embedded system, to name a few non-limiting examples, or any combination thereof.

Computer system 400 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 400 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 400, main memory 408, secondary memory 410, and removable storage units 418 and 422, as well as tangible articles of manufacture embodying any combination of the foregoing. Such control logic, when executed by one or more data processing devices (such as computer system 400), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 4. In particular, embodiments can operate with software, hardware, and/or operating system implementations other than those described herein.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

What is claimed is:

1. A system for determining a digital phenotype of a target individual, the system comprising:
a data processing handler configured to receive a data stream, wherein the data stream comprises at least one of: audio data of the target individual, visual data depicting the target individual, and text data comprising at least one of: a health record of the target individual, an audio recording transcription, and a textual note;
a machine learning model configured to:
receive as input the at least one of the audio data, the visual data, and the text data;
determine, dependent on receipt of the visual data, a first output estimation comprising at least one of: a facial action unit intensity, or a valence and arousal estimation pair;
determine, dependent on receipt of the audio data, a second output estimation comprising at least one of: an emotion classification or a voice prosody feature;
determine, dependent on receipt of the visual data, a third output estimation comprising at least one of: a heart rate of the target individual, a raw blood volume pulse signal of the target individual, or a heart rate variability of the target individual;
determine, dependent on receipt of the text data, a fourth output estimation comprising at least one of: an image and prompt correlation utilizing the health record of the target individual or the textual note, and a sentiment analysis of the audio recording transcription;
determine a digital phenotype based on at least one of: the first output estimation, the second output estimation, the third output estimation, and the fourth output estimation; and
an output handler configured to output the digital phenotype.

2. The system of claim 1, wherein the machine learning model comprises a plurality of machine learning models.

3. The system of claim 1, wherein the data stream further comprises biometric data of the target individual and wherein the machine learning model is further configured to determine, dependent on receipt of the biometric data, a fifth output estimation highlighting anomalies in the biometric data.

4. The system of claim 3, wherein the anomalies are highlighted based on a comparison to an estimated baseline of the target individual.

5. The system of claim 3, wherein the biometric data is generated by a sensor device.

6. The system of claim 1, wherein the data stream further comprises at least one of: contact sensing data of the target individual or physiological data of the target individual.

7. The system of claim 6, wherein the physiological data is received from a sensor associated with the target individual.

8. The system of claim 1, wherein the first output estimation further comprises at least one of: data representing a face of the target individual, data representing a body of the target individual, or data representing a pose of the target individual.

9. The system of claim 1, wherein the machine learning model comprises at least one of: a neural network configured to perform a neural network method, a deep neural network, a transformer model, a recurrent neural network-based model, or a large language model.

10. The system of claim 9, wherein the transformer model is configured to employ a probsparse method or a self-attention method.

11. The system of claim 1, wherein the machine learning model is further configured to execute a zero-shot contrastive pre-training method.

12. The system of claim 1, wherein the machine learning model is further configured to:
determine a first intermediate representation of at least one of: an emotional affect of the target individual using the visual data or a body of the target individual using the visual data;
determine a second intermediate representation of a voice of the target individual using the audio data;
determine a third intermediate representation of at least one of: a face of the target individual using the visual data or a body of the target individual using the visual data; and
determine a fourth intermediate representation of least one of: the health record of the target individual using the text data, biometric data using the text data, or the image and prompt correlation.

13. The system of claim 12, wherein the machine learning model is further configured to:
receive as input at least one of: the first intermediate representation, the second intermediate representation, the third intermediate representation, or the fourth intermediate representation; and
determine, based on the received input and by applying a weight or transform logic, at least one of: an output time series forecast or a data imputation.

14. The system of claim 13, wherein determining the digital phenotype is further based on at least one of: the first intermediate representation, the second intermediate representation, the third intermediate representation, fourth intermediate representation, the output time series forecast, or the data imputation.

15. The system of claim 1, wherein to output the digital phenotype, the output handler is configured to perform at least one of:
transmitting the digital phenotype to a computing device or a remote display platform;
displaying the digital phenotype as a visual notification; or
storing the digital phenotype in a memory location.

16. The system of claim 15, wherein the output handler is further configured to transmit the digital phenotype, display the digital phenotype, or store the digital phenotype, during at least one of: a psychiatric session or a telehealth session.

17. The system of claim 16, wherein the output handler is further configured to summarize the digital phenotype and provide the summary to a medical practitioner of the psychiatric session or a medical practitioner of the telehealth session.

18. The system of claim 17, wherein the summarized digital phenotype is represented as a numerical representation or a textual representation.

19. The system of claim 15, wherein the output handler is further configured to display the digital phenotype in a graphical user interface.

20. The system of claim 15, wherein the memory location corresponds to an electronic health record, and wherein the digital phenotype is added to the electronic health record.

21. The system of claim 15, wherein the output handler is further configured to transmit the digital phenotype, display the digital phenotype, or store the digital phenotype, during a coaching session for a neurodiverse population.

22. The system of claim 15, wherein the output handler is further configured to transmit the digital phenotype, display the digital phenotype, or store the digital phenotype, during a telehealth or in-person assessment of individuals with neurological or developmental disorders/conditions.

23. The system of claim 15, wherein the output handler is further configured to display the digital phenotype as a textual guidance or a visual guidance for socio-behavioral learning or job coaching.

24. The system of claim 1, wherein the output handler is further configured to output the digital phenotype in an audio format.

25. The system of claim 1, wherein the digital phenotype includes a confidence score.

26. The system of claim 25, wherein the output handler is further configured to display the confidence score.

27. The system of claim 26, wherein the output handler is configured to display the confidence score based on determining the confidence score is less than a predefined threshold.

28. The system of claim 25, wherein the output handler is configured output the digital phenotype based on determining the confidence score is greater than a predefined threshold.

29. The system of claim 1, wherein the machine learning model is a layer within a plurality of layers of a second machine learning model.

30. The system of claim 1, wherein the digital phenotype comprises at least one of: an emotion estimate, a behavior prediction, a sub-clinical biomarker estimate, a mood disorder state of the target individual within a Depression, Anxiety, and Stress scale (DASS), a Patient Health Questionnaire (PHQ-9) estimate, a Generalized Anxiety Disorder (GAD-7) estimate, or a distress warning sign.

31. The system of claim 30, wherein the emotion estimate includes at least one of: happy, angry, sad, neutral, delighted, excited, tense, angry, frustrated, depressed, bored, tired, calm, relaxed, or content.

32. The system of claim 30, wherein the behavior prediction includes a trendline of quantitative biomarker estimates or an associated interpretation statement.

33. The system of claim 30, wherein the digital phenotype further comprises at least one of: a predicted heart rate of the target individual, a predicted raw blood volume pulse signal of the target individual, or a predicted heart rate variability of the target individual.

34. A system for determining a digital phenotype of a target individual, the system comprising:
a data processing handler configured to receive a data stream, wherein the data stream comprises at least one of:
audio data comprising a vocal feature of the target individual,
visual data comprising at least one of: an image of a face of the target individual, an image of a body of the target individual,
contact sensing data of the target individual,
physiological data of the target individual, and
text data comprising a health record of the target individual;
a first machine learning model is configured to:
receive as input, the visual data from the data processing handler; and
extract the face of the target individual;
wherein the first machine learning model comprises at least one of: a neural network configured to perform a neural network method, a deep neural network, or a transformer model;
a second machine learning model configured to:
receive as input the visual data from the data processing handler;
determine a first intermediate representation of at least one of: an emotional affect of the target individual using the visual data or the body of the target individual using the visual data; and
determine an output estimation comprising at least one of: a facial action unit intensity, or a valence and arousal estimation pair;
wherein the second machine learning model comprises at least one of: a neural network configured to perform a neural network method, a deep neural network, or a transformer model;
a third machine learning model configured to:
receive as input the audio data from the data processing handler;
determine a second intermediate representation of a voice of the target individual using the audio data; and
determine an output estimation comprising at least one of: an emotion classification or a voice prosody feature,
wherein the third machine learning model comprises at least one of: a neural network configured to perform a neural network method, a deep neural network, or a transformer model;
a fourth machine learning model configured to:
receive as input the visual data from the data processing handler; and
determine a third intermediate representation of at least one of: the face of the target individual using the visual data or the body of the target individual using the visual data;
determine an output estimation comprising at least one of: a heart rate of the target individual, a raw blood volume pulse signal of the target individual, or a heart rate variability of the target individual,
wherein the fourth machine learning model comprises at least one of: a neural network configured to perform a neural network method, a deep neural network, or a transformer model;
a fifth machine learning model configured to:
receive as input at least one of: the visual data from the data processing handler or the text data from the data processing handler; and
determine a fourth intermediate representation of least one of:
the health record of the target individual using the text data or biometric data using the text data, or
an image and prompt correlation of at least one of: the health record of the target individual using the visual and text data or biometric data using the visual and text data,
wherein the fifth machine learning model is configured to execute a zero-shot contrastive pre-training method;
a sixth machine learning model configured to:
receive as input at least one of: the first intermediate representation, the second intermediate representation, the third intermediate representation, or the fourth intermediate representation; and
determine, based on the received input and by applying a weight or transform logic, at least one of: an output time series forecast or a data imputation, and
wherein the sixth machine learning model comprises at least one of: a neural network configured to perform a neural network method, a recurrent neural network-based model, a transformer model configured to employ a probsparse method or a self-attention method, or a large language model;
a seventh machine learning model configured to:
receive as input at least one of: the first intermediate representation, the second intermediate representation, the third intermediate representation, the fourth intermediate representation, the output time series forecast, or the data imputation; and
determine a digital phenotype;
an output handler configured to:
receive as input at least one of: the first intermediate representation, the second intermediate representation, the third intermediate representation, the fourth intermediate representation, the output time series, the data imputation, the contact sensing data, the biometric data, or the digital phenotype; and
perform at least one of:
transmitting the digital phenotype to a computing device or a remote display platform;
displaying the digital phenotype as a visual notification; or
storing the digital phenotype in a memory location.

35. A method for determining a digital phenotype of a target individual, the method comprising:
receiving, by a data processing handler, a data stream,
wherein the data stream comprises at least one of: audio data of the target individual, visual data depicting the target individual, and text data comprising at least one of: a health record of the target individual, an audio recording transcription, and a textual note;
receiving as input, by a machine learning model, at least one of the audio data, the visual data, and the text data;
determining, dependent on receipt of the visual data and by the machine learning model, a first output estimation comprising at least one of: a facial action unit intensity, or a valence and arousal estimation pair;
determining, dependent on receipt of the audio data and by the machine learning model, a second output estimation comprising at least one of: an emotion classification or a voice prosody feature;
determining, dependent on receipt of the visual data and by the machine learning model, a third output estimation comprising at least one of: a heart rate of the target individual, a raw blood volume pulse signal of the target individual, or a heart rate variability of the target individual;
determining, dependent on receipt of the text data and by the machine learning model, a fourth output estimation comprising at least one of: an image and prompt correlation utilizing the health record of the target individual or the textual note, and a sentiment analysis of the audio recording transcription;
determining, by the machine learning model, a digital phenotype based on at least one of: the first output estimation, the second output estimation, the third output estimation, and the fourth output estimation; and
outputting, by an output handler, the digital phenotype.
